(19) United States
(12) Patent Application Publication
(10) Pub. No.: US 2019/0105468 A1
(43) Pub. Date: Apr. 11, 2019

(54) MEDICAL CONTINUUM ROBOT WITH MULTIPLE BENDABLE SECTIONS

(71) Applicants: Canon U.S.A., Inc., Melville, NY (US); The Brigham and Women's Hospital Inc., Boston, MA (US)

(72) Inventors: Takahisa Kato, Brookline, MA (US); Yohei Minatoya, Syosset, NY (US); Yusuke Tanaka, Tokyo (JP); Kiyoshi Takagi, Tokyo (JP); Oliver Jonas, Weston, MA (US)

(21) Appl. No.: 16/152,317

(22) Filed: Oct. 4, 2018

Related U.S. Application Data
(60) Provisional application No. 62/568,683, filed on Oct. 5, 2017.

(51) Int. Cl. A61M 25/01; A61B 6/03; A61B 10/06

(52) U.S. Cl. CPC A61M 25/0113; A61M 2210/1039; A61B 10/06; A61B 6/032

(57) ABSTRACT

The subject disclosure is directed to an articulated medical device having a hollow cavity, wherein the device is capable of maneuvering within a patient, and allowing a medical tool to be guided through the hollow cavity for medical procedures, including endoscopes, cameras, and catheters.

### CROSS REFERENCE TO RELATED PATENT APPLICATIONS

This application claims priority from U.S. Provisional Patent Application No. 62/568,683 filed on Oct. 5, 2017, in the United States Patent and Trademark Office, the disclosure of which is incorporated herein in its entirety by reference.

### FIELD OF DISCLOSURE

The present disclosure relates generally to apparatus and methods for medical application. More particularly, the subject disclosure is directed to an articulated medical device having a hollow cavity, wherein the device is capable of maneuvering within a patient, and allowing a medical tool to be guided through the hollow cavity for medical procedures, including endoscopes, cameras, and catheters.

### BACKGROUND OF THE DISCLOSURE

Articulated sheaths generally include one or more channels that extend along the inside of the sheath to allow access to end effectors (the actual working part of a surgical instrument or tool) located at a distal end of the sheath. Control mechanisms located at a proximal end of the sheath are configured to enable remote manipulation of the end effectors via the one or more channels. Accordingly, the mechanical structure of the sheath plays a key role in ensuring flexible access to end effectors, while protecting delicate organs and tissues of a patient.

End effectors may include clamps, graspers, scissors, staplers, needle holders, and other like tools, which can manipulate body parts (organs or tissue) during examination or surgery. Also, some sheaths (e.g., endoscopic instruments) include a light delivery system that can illuminate a body part under inspection and an imaging system that observes the body part under inspection.

The lack of steering ability of current manual catheter for various medical procedures (e.g. transbronchial biopsy in the lung) severely hampers a physician's ability to reach a remote area (e.g. nodule in peripheral lung) which ultimately reduces the performance and efficacy of the procedure.

Accordingly, there exists a need in the art for a medical apparatus that is diminutive in size and capable of multiple bends to maneuver through various cavities to reach a remote area. The subject disclosure provides an improvement of the maneuverability of the robotic catheter, in addition to a greater aptitude to reach a peripheral area cavities in a subject.

### SUMMARY

Thus, to address such exemplary needs in the industry, the present disclosure teaches apparatus, systems and method for a robotic catheter capable of adept maneuverability at a greater aptitude and precision, to reach peripheral areas within a subject.

In various embodiments, the subject disclosure teaches a medical apparatus comprising: a driving unit; a single sheath that includes a first bendable segment, a second bendable segment, and a third bendable segment, which are bendable by the driving unit; and a controller configured to send a control signal to the driving unit for bending the at least three bendable segments.

In one embodiment, the apparatus has a length ratio between the first bendable segment and second bendable segment which is between 1:1 to 1:5.

In other embodiments, the medical apparatus teaches the first bendable segment and the second bendable segments to be distal to the third bendable segment, and the first bendable segment and the second bendable segment are configured to be three-dimensionally bent by the driving unit.

In yet additional embodiment, the third bendable segment may be at least twice as long as each of the first and the second bendable segments, and the third bendable segment may be deformable by external force so that the third bendable segments can have a shape with at least one inflection point.

The subject disclosure further discloses the driving unit being configured to apply a force to the third bendable segment in order to bend or maintain an orientation of a distal end of the third bendable segment while the external force is applied.

In further embodiments, the subject disclosure iterates wherein the controller is configured to control the first bendable segment and the second bendable segment independently of the third bendable segment, while a force is applied from the controller to the third bendable segment via the driving unit in order to maintain a shape of the third bendable segment.

In another variant of the subject disclosure, the length of the first bendable segment and the second bendable segment is less than one tenth of the length of the third bendable segment.

In further embodiments, the length of the first bendable segment is less than the length of the second bendable segment.

In another embodiment, a length ratio between the second bendable segment and third bendable segment is between 1:2 to 1:20.

Another embodiment of the subject disclosure teaches a length ratio between the first, the second, and the third bendable segments as 1:2:17, respectively.

In further embodiments, the diameters of the first bendable segment and the second bending segment are identical.

Further disclosure of the subject innovation includes the first bendable segment and the second bendable segment being configured to independently change a respective bending angle and a respective bending plane in a three dimension space.

In additional embodiments, the controller is configured to dislocate the distal end of the single sheath three-dimensionally while keeping a substantially constant orientation of the distal end of the single sheath by bending the first bendable segment and the second bendable segment.

Further embodiments contemplate the sheath further comprises an outer wall covering at least the first bendable segment, wherein the outer wall is configured to attach to the first bendable segment and provide flexible support to the sheath.

In other embodiments, the first bendable segment comprises a plurality of guide rings distributed along the longitudinal direction of the sheath, wherein the plurality of guide rings distributed along the longitudinal direction of the sheath create cavities between each of the guide rings. In further embodiments, the plurality of guide rings distributed along the longitudinal direction of the sheath are flexibly supported by an outer wall.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 illustrates an example embodiment of a continuum robot system.

FIG. 2 illustrates a side view of an example embodiment of a sheath.

FIG. 3A illustrates the guide rings and the driving wires of an example embodiment of a sheath.

FIG. 3B illustrates the guide rings and the driving wires of an example embodiment of a sheath.

FIG. 4 is a schematic drawing that illustrates the bending angle and the bending plane of each bendable segment.

FIG. 5 illustrates the maximum bending angle of a bending segment.

FIGS. 6(a)-(e) illustrate the rotation of the bending plane along the centroid of an example embodiment of the first bendable segment 12.

FIG. 7 is a side perspective cut-out view of one embodiment of a bendable body, according to the subject disclosure.

FIG. 8A is a schematic drawing that explains the targeting of a lesion in a peri-bronchial area.

FIG. 8B illustrates an example of omni-directional orientation.

FIG. 9. illustrates an example embodiment of cluster sampling.

FIGS. 10A and 10B illustrate an advantage of cluster sampling.

FIGS. 11A-B illustrate examples of cluster sampling.

FIG. 12 shows an example embodiment of a sheath navigating through a bifurcation point.

FIG. 13 is a side perspective view of a continuum robot system, according to one or more embodiment of the subject disclosure.

FIG. 14A is a cut-out view of a model of a right upper lobe of the lung, illustrating various paths for advancement of a continuum robot system, according to one or more embodiment of the subject disclosure.

FIG. 14B is an illustration of a right upper lobe of the lung, identifying various cavities for advancement of a continuum robot system, according to one or more embodiment of the subject disclosure.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

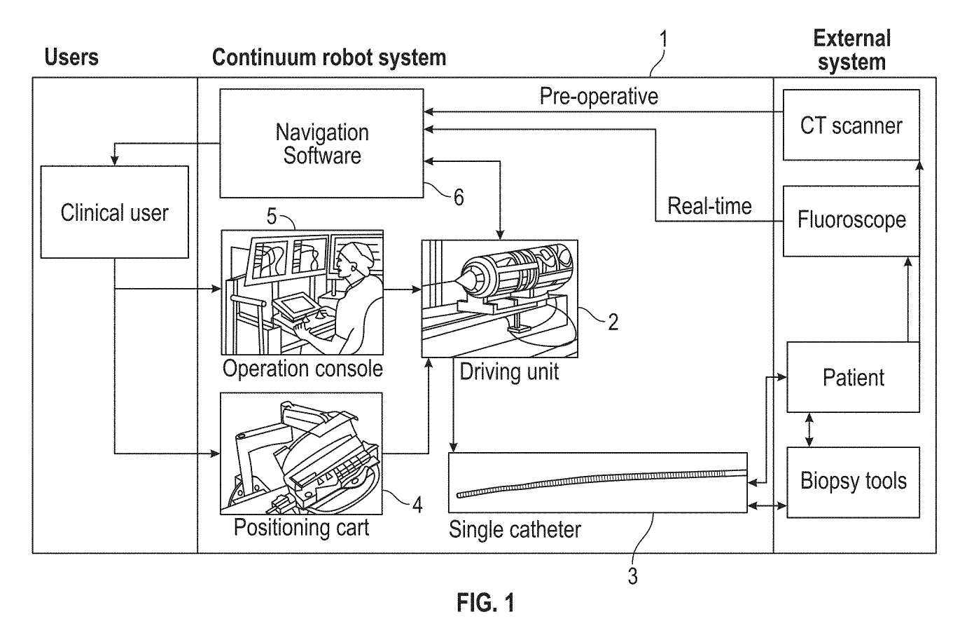

FIG. 1 illustrates an example embodiment of a continuum robot system. The continuum robot system 1 comprises a driving unit 2, a sheath 3, a positioning cart 4, an operation console 5, and navigation software 6. The system 1 also interacts with clinical users and external systems (e.g., a CT scanner, a fluoroscope, a patient, biopsy tools).

The navigation software 6 and the driving unit 2 are communicatively coupled via a bus, which transmits data between them. Moreover, the navigation software 6 is coupled to and communicates with a CT scanner, a fluoroscope, and an image server (not in FIG. 1), which are external of the continuum robot system 1. The image server may be, for example, a DISCOM server that is coupled to a medical imaging device, such as a CT scanner, a MRI scanner, and a fluoroscope. The navigation software 6 processes data provided by the driving unit 2, data provided by images stored on the image server, images from the CT scanner, and images from the fluoroscope in order to display images on a display device.

The images from the CT scanner are pre-operatively provided to the navigation software 6. With the navigation software 6, a clinical user can create an anatomical computer model from the images. In some embodiments, the anatomy is lung airways. From the chest images of the CT scanner, the clinical user can segment the lung airways for clinical treatments, such as a biopsy.

After generating the lung-airway map, the clinical user can also create a plan to access the lesion for a biopsy. The plan includes the airways to insert the single sheath 3 and the lesion.

The driving unit 2 comprises actuators and a control circuitry. The control circuitry is communicatively-coupled with the operation console 5. Also, the driving unit 2 is connected to the single sheath 3 so that the actuators in the driving unit 2 operate the single sheath 3. Therefore, a clinical user can control the single sheath 3 via the driving unit 2.

The driving unit 2 is also physically connected to a positioning cart 4. The positioning cart 4 includes a positioning arm, and the positioning cart 4 locates the driving unit 2 and the single sheath 3 in the intended position against a patient. The clinical user can insert and retreat the sheath 3 to perform a biopsy in the airways of the patient.

The sheath 3 can be navigated to the lesion in the airways based on the plan by the clinical user's operation. The sheath 3 includes a tool channel for a biopsy tool. The sheath 3 can guide the biopsy tool to the lesion of the patient. The clinical user can take a biopsy sample from the lesion with the biopsy tool.

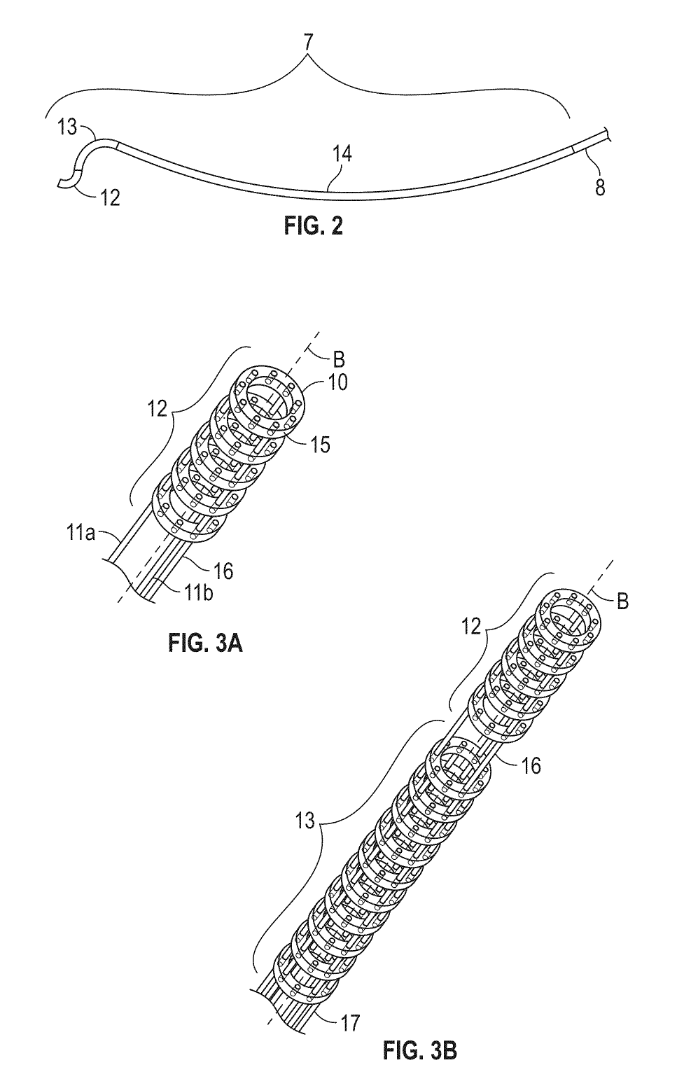

FIG. 2 is a side view of an example embodiment of a sheath. The sheath comprises a robotized part 7, a proximal flexible part 8, and a connection hub 9.

The sheath 3 also includes a tool channel through the robotized part 7, the proximal flexible part 8, and the connection hub 9. The tool channel has a substantially-cylindrical configuration. The clinical user can insert and retrieve a biopsy tool through the tool channel from the connection hub 9 to the distal end of the robotized part 7.

The connection hub 9 is mounted on the driving unit 2 and connects driving wires 11 to the actuators in the driving unit 2.

The robotized part 7 includes multiple guide rings 10 and multiple driving wires 11. The guide rings 10 are arrayed by identical intervals, and the guide rings 10 hold the driving wires 11 that are slide-able along longitudinal direction of the robotized part 7. The driving wires 11 are also terminated on the ring guides 10 in some embodiments (different driving wires 11 may terminate on different ring guides 10), and the driving wires 11 can bend the robotized part 7 in three different segments. Thus, the robotized part 7 forms a first bendable segment 12, a second bendable segment 13, and a third bendable segments 14, as show in FIG. 2.

The proximal flexible part 8 includes the driving wires 11 from the robotized part 7 to the connection hub 9 and transmits the driving force from the actuators in the driving unit 2 to the robotized part 7.

The robotized part 7 and the proximal flexible part 8 may have identical outer diameters (e.g., 3 mm) and identical inner diameters (e.g., 1.8 mm). The inner diameters form the tool channel. In some embodiments, the lengths of the robotized part 7 and the flexible part 8 are 200 mm and 600 mm, respectively.

FIG. 3A illustrates the guide rings and the driving wires of an example embodiment of a sheath. FIG. 3A shows an exploded view of the first bendable segment 12. The first bendable segment 12 includes five ring guides 10, two driving wires 11a-11b, and a fixed wire 16. Also, FIG. 3A indicates anchors 15 with red dots.

In this embodiment, the ring guides 10 are equidistantly arrayed along a centroid B with 2 mm intervals by attaching all five ring guides 10 to the fixed wire 16 with five respective anchors 15. Therefore, the fixed wire 16 is terminated to five guide rings 10 in the first bendable segment 12.

On the other hand, the driving wires 11a and 11b are terminated on the guide ring 10 at a distal end of the first bendable segment 12, but not on the other four guide rings 10. By pushing and pulling the driving wires 11a and 11b, while holding the first fixed wire 16 in a fixed position, the first bendable segment 12 can bend three-dimensionally.

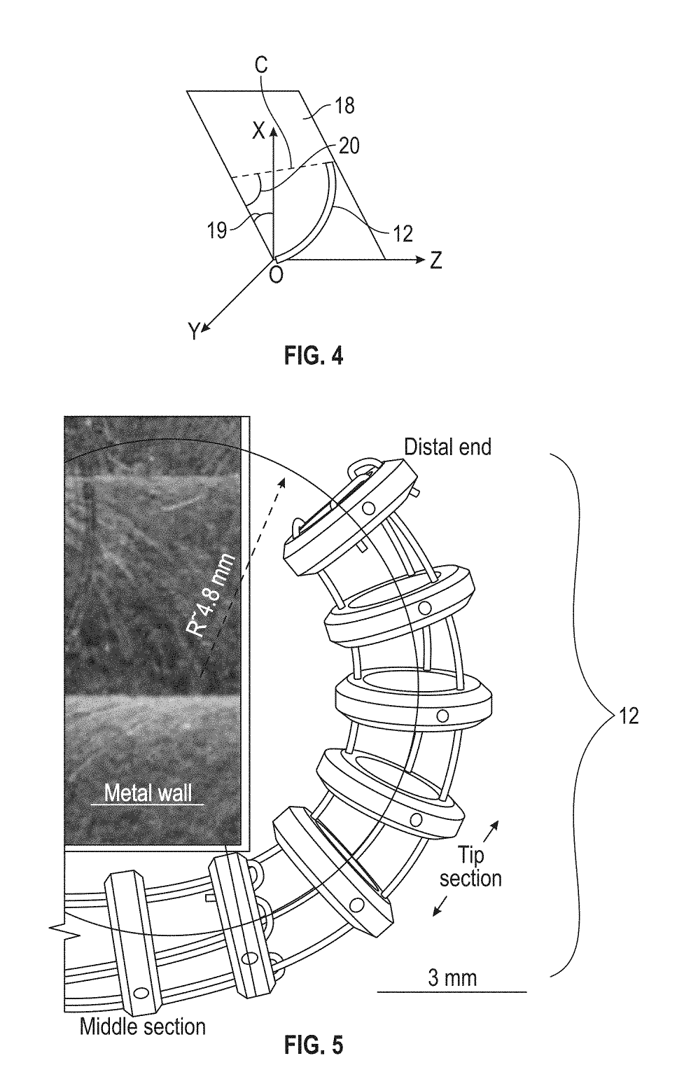

FIG. 4 is a schematic drawing that illustrates the bending angle and the bending plane of each bendable segment. FIG. 4 shows the three-dimensional bending of the first bendable segment 12. The coordinate O-X-Y-Z is the local coordinate of the first bendable segment 12. The origin of this coordinate is at the proximal end of the first bendable segment 12 on the centroid B. The Z axis is aligned to the centroid B. The three-dimensional bending of the first bendable segment 12 is defined by a bending angle 20 and a bending-plane direction 19 on a bending plane 18. Similar local coordinates can be defined for the second and the third bendable segments 13 and 14, which are show in FIG. 2.

In FIG. 4, the first bendable segment 12 bends with one constant curvature in three-dimensional space. The bending plane 18 is a plane that includes the bended shape of the first bendable segment 12. The bending plane 18 also includes the Z axis and is perpendicular to the X-Y plane. In FIG. 4, the bending-plane direction 19 is an angle between the X axis and the bending plane 18.

The bending angle is an angle that is defined on the bending plane 18. Line C is perpendicular to the centroid B and is tangential to the distal end of the first bending segment 12 on the bending plane 18. The bending angle 20 is the angle between the X-Y plane and the line C.

The second and the third bendable segments 13 and 14 also include a similar termination structure of the guide rings 10, the driving wires 11 and the first fixed wire 16. FIG. 3B, which illustrates the guide rings and the driving wires of an example embodiment of a sheath, shows this termination structure in the second bendable segment 13. The second bendable segment 13 includes another two driving wires and a second fixed wire 17. These two driving wires are terminated at the distal end of the second bendable segment 13. Also, the second fixed wire 17 is terminated in all of the guide rings 10 that are in the second bendable segment 13, and the second fixed wire 17 forms the equidistant array of the guide rings 10, for example in intervals of 2 mm. The guide rings 10 in the second bendable segment 13 also include the guide holes for the driving wires 11a-11b and the first fixed wire 16 for the first bendable segment 12. Those guide holes mechanically hold the driving wires 11a-11b and the first fixed wire 16 for the first bendable segment 12 while those wires are slide-able along the centroid B of the second bendable segment 13. The third bendable segment 14 has a similar configuration against the first bendable segment 12 and the second bendable segment 13. Therefore, by pushing and pulling the driving wires 11, the first bendable segment 12, the second bendable segment 13, and the third bendable segment 14 can individually bend three-dimensionally (i.e., each bendable segment can bend independently of the other bendable segments).

FIG. 5 illustrates the maximum bending angle of a bending segment. The plane of FIG. 5 is aligned to the bending plane 18. The first bendable segment 12 can bend while the second bendable segment 13 stays straight on the bending plane. This particular embodiment of the first bendable segment 12 can bend with a curvature radius of approximately 5 mm.

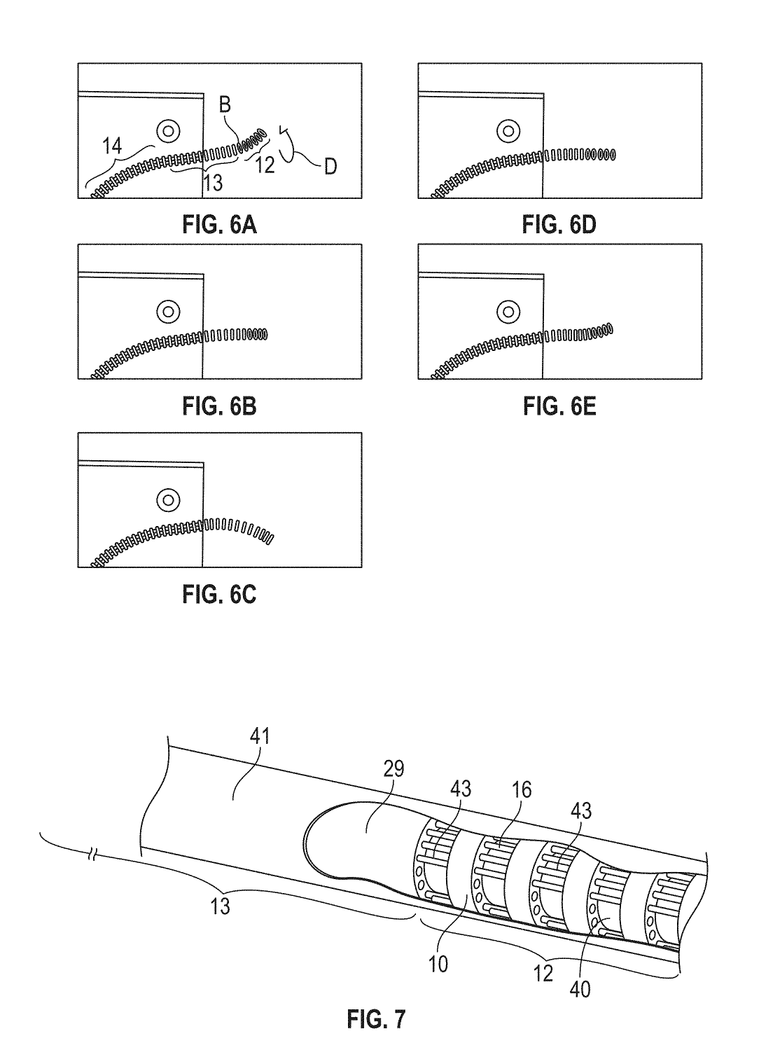

FIGS. 6(a)-(e) illustrate the rotation of the bending plane along the centroid of an example embodiment of the first bendable segment 12. The line B is the centroid of the first bendable segment 12, which may be identical to the Z axis of the local coordinate system of the first bendable segment.

In this embodiment, the first bendable segment bends at around 3o degrees on the bending plane on the same plane as the plane of the page, as shown in FIG. 6(a). After that, the sheath 3 rotates the bending plane 18 along the path shown by the arrow D, as shown in FIGS. 6(b) to 6(e). During this rotation, the sheath 3 can rotate the bending plane of the first bendable segment 12 without moving the second bendable segment 13 and without moving the third bendable segment 14. Additionally, the third bendable segment 14 can also be deformed by an external force.

To elaborate, the driving unit 2 bends the third bendable segment 14 to orient the distal end of the third bendable segment 14 to the direction of the channel. Because the channel has one curvature in the place where the third bendable segment 14 is located, the force bending the third bendable segment 14 is mainly an internal force from the driving wires 11. As the sheath 3 advances into the channel, the third bendable segment 14 enters another curvature in the channel. Although the driving unit 2 still continues to bend the third bendable segment 14 to orient the distal end of the third bendable segment 14 to the direction of the channel, the entire shape of the third bendable segment 14 follows the S-shaped channel and forms an inflection point 21 at a boundary between the two curvatures. The third bendable segment 14 contacts with the channel and is deformed at the S-shaped by the external force from the interaction between the channel and the sheath.

FIG. 7 provides a cut-out view of the second bendable segment 13 and the first bendable segment 12 of the subject continuum robot system 1, according to one or more embodiment of the subject disclosure.

In this particular embodiment, the bendable segments 12 and 13 have an outer diameter of 3 mm and an inner diameter of 2 mm. Also, the tip of the robotized part 7 may be rounded to prevent damage when advanced.

The first fixed wire 16 is slide-ably guided through the guide ring 10, with anchor 15 at the end to anchor to the last guide ring 10, the first fixed wire 16 is constrained by the robotized part 7. Therefore, the first fixed wire 16 can transmit pushing, pulling forces and torque to the robotized part 7 to actuate the first bendable segment 12. In the same manner, the second fixed wire 17 can bend the second bendable segment 13. The first and second bendable segment 12 and 13 also include cylindrical inner and outer walls 40 and 41, respectively. Those walls are attached to each guide ring 10 and create cavities distributed along the longitudinal direction A. Those cavities create evenly distributed wrinkling shape in both cylindrical inner and outer walls 40 and 41 when the first and second bendable segments bend at various curvatures. Therefore, the cavity can avoid fatal kinking which may crush the tool channel 42 even when the bendable segments 12 and 13 include thin total wall thickness.

The robotized part 7 also has support wires 43. The support wire 43 is terminated at the distal end of the robotized part 7 and goes thorough satellite lumen 28 to the proximal end. Since the support wire 43 is configured in parallel to longitudinal direction A between fixed wires 16 and 17, the support wire 43 would not increase total wall thickness. At the same time, the support wire 43 would further prevent fatal kinking by providing an elastic element between guide rings 10.

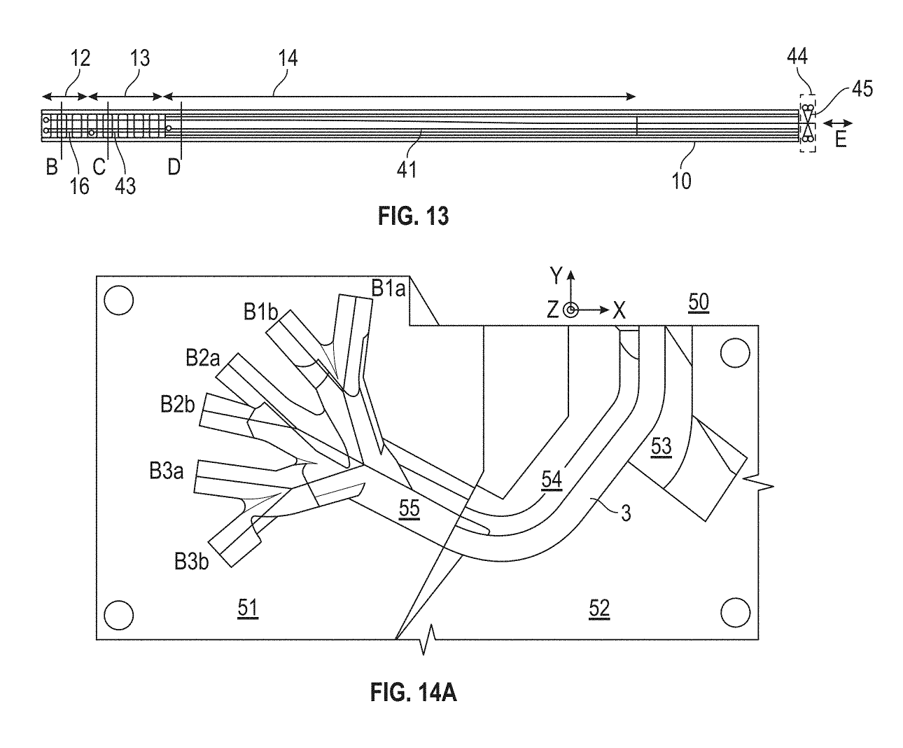

FIG. 13 is the schematic cross sectional view of the robotized part 7. The proximal end of the support wire 43 is attached proximal termination structure 44. The proximal termination structure 44 is a slider element 45 that supports the support wire 43 slide-ably. Therefore, the support wire 43 would not be subjected to the tension and contraction forces when the robotized part 7 is bended, and minimize additional bending rigidity. The support wires 43 is elastically terminated at the proximal termination structure 44, and provide restoring force without increasing wall thickness.

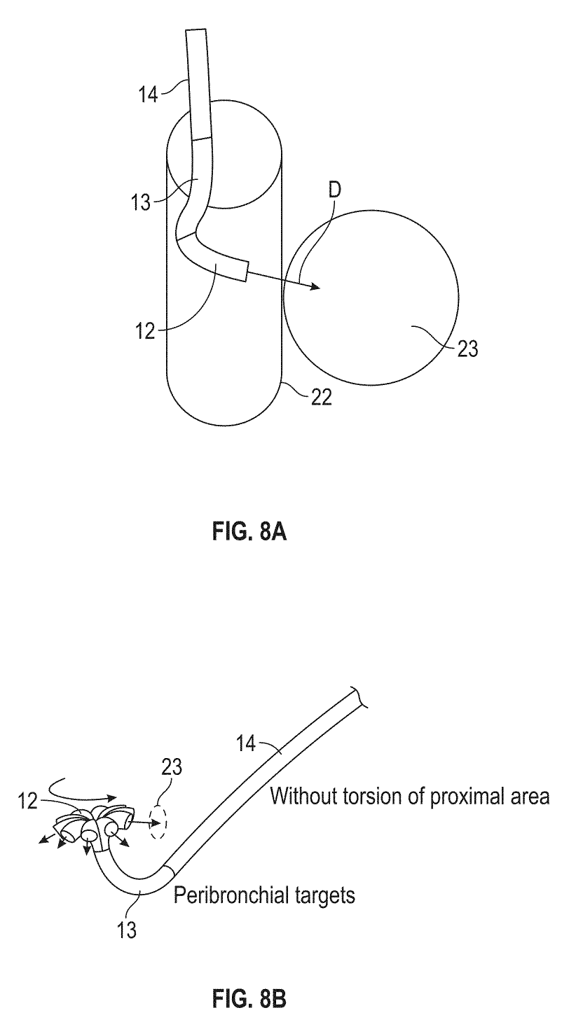

FIG. 8A is a schematic drawing that explains the targeting of a lesion in a peri-bronchial area, which is a lateral area surrounding the airways. Targeting this lesion can be challenging because of the limited distal dexterity of a conventional sheath.

In FIG. 8A, the sheath 3 advances in the airway 22 through bifurcations of the airway and directs the distal end (which is also an end of the first bendable segment 12) to the lesion 23 by bending the first bendable segment 12 and the second bendable segment 13. Because the airway doesn't directly connect with the lesion 23, this is one of the difficult configurations for a conventional sheath. With the first bendable segment 12, the second bendable segment 13, and the third bendable segment 14, the sheath 3 can orient the distal end without moving the proximal part of the sheath that passes through all the airway's bifurcations to the lesion 23.

Thus, by using the three-dimensional bending capability of the first bendable segment 12 and the second bendable segment 13, the sheath can perform difficult maneuvers to enhance capability of the peri-bronchial targeting.

For example, one maneuver is omni-directional orientation, which is shown in FIG. 8B. The first bendable segment 12 can rotate without rotating any other part of the sheath 3. This maneuver is beneficial to determine the orientation of the distal end to the lesion 23 because this motion isn't affected by the physical interaction of the proximal part of the sheath to the anatomy and because this motion doesn't affect the position of the lesion while the sheath physically maps the orientation of the distal end with robotic control. Moreover, with the second bendable segment 13, the sheath 3 can perform this omni-directional orientation after passing through a final bifurcation point on its way to the lesion 23.

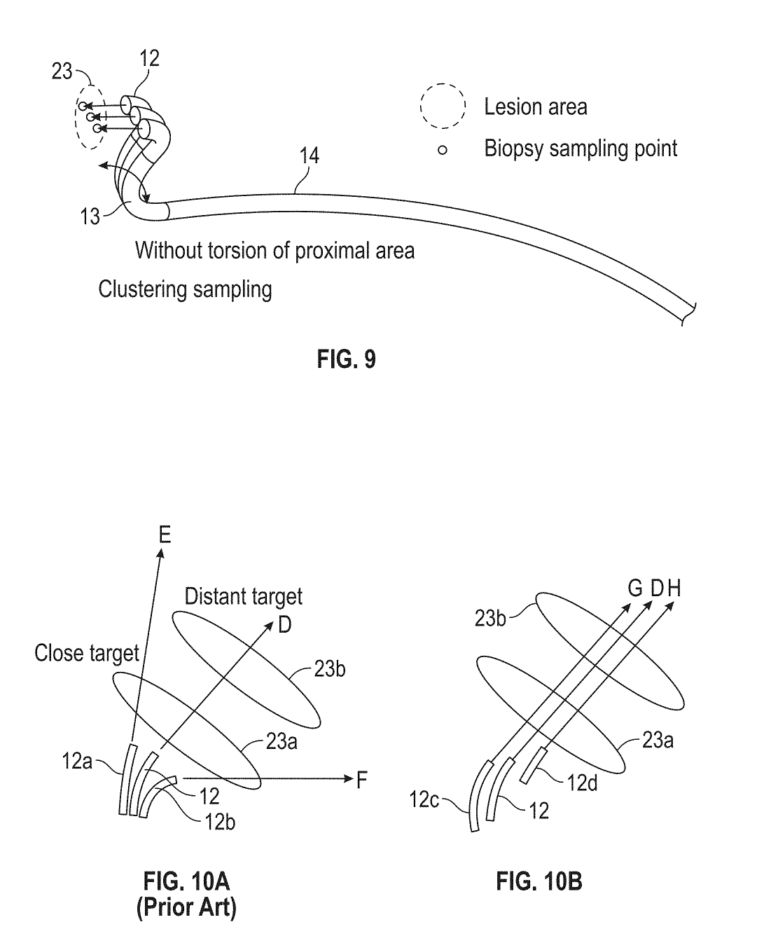

Another maneuver is cluster sampling, which is shown in FIG. 9. The first bendable segment 12 can dislocate the position of the distal end while maintaining the orientation of the distal end. With this maneuver, the distal end can access different positions of the lesion 23.

An advantage of cluster sampling is illustrated in FIGS. 10A and 10B. With a conventional sheath (FIG. 10A), to access different position in the lesion 23a or to perform fine adjustments of the position of the distal end, the distal end of a steerable sheath can bend or the distal end can rotate by torqueing the proximal end of the sheath (a pre-curved part of the sheath). With either one of these approaches, the distal end needs to change its orientation to change the access position, for example as shown by arrows D, E, and F in FIG. 10A. Therefore, a factor in the resolution, accuracy, and precision of the positioning is a distance to the lesion, and the resolution, accuracy, and precision deteriorate as the distance increases.

On the other hand, the sheath 3 can dislocate the distal end while maintaining the same orientation, as shown in FIG. 10B. Therefore, the resolution, accuracy, and precision of the positioning are maintained through the dislocation of the distal end.

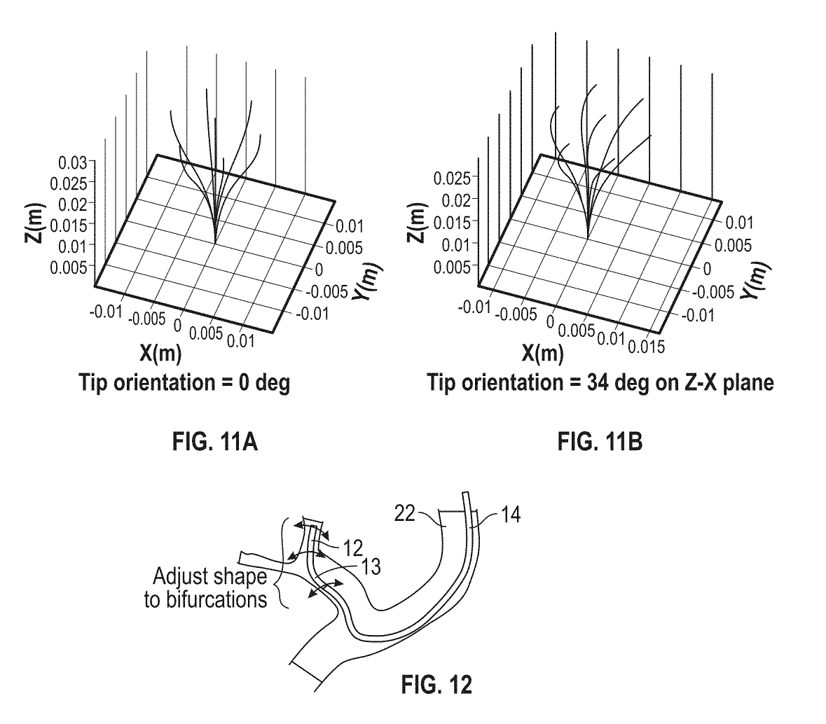

FIGS. 11A-B illustrate examples of cluster sampling. FIGS. 11A-B show the posture of the first bendable segment 12 and the second bendable segment 13 at the local coordinates of the second bendable segment 13. The lines though the center are the center lines of those two bendable segments. The different colors show different targeting positions that have the same distal orientation.

In some embodiments, the lengths of the first bendable segment 12 and the second bendable segment 13 are, respectively, 10 and 20 mm. In FIG. 11A, the original shape is the straight pose. The distal end can dislocate around a circumference that has a diameter of 10 mm while maintaining the same orientation, as shown by the other six poses.

Also, as shown by FIG. 11B, the distal end can perform the same maneuver to dislocate around a circumference with a diameter of 10 mm with a different original shape. In FIG. 11B, the original shape is the shape with the first bendable segment bent at 34 degrees. With this original shape, the sheath can also derive the other six shapes by dislocating the distal end around the circumference while keeping the original orientation.

The first bendable segment and the second bendable segment can also be used to navigate the sheath 3 through a bifurcation point, as shown by FIG. 12. The first bendable segment 12 can adjust the shape or orientation to the daughter branch while the second bendable segment 13 can adjust the shape or orientation to the parent branch in the bifurcation point, as the sheath 3 advances through the bifurcation point. Once the first bendable segment 12 and the second bendable segment 13 pass the bifurcation point, those parts can become guiding parts for the rest of the proximal part, so that the insertion force from the proximal end of the single sheath can be effectively transformed into the insertion force for distal parts of the sheath without serious looping.

Thus, embodiments of the continuum robot can enhance the distal dexterity with the first bendable segment and the second bendable segment. The first and second bendable segments, which are distal to the third bendable segment, can change the orientation of the distal end of the sheath, as well as the position of the distal end, three-dimensionally. With those motions, the distal end (after the third bendable segment) of the sheath can access a wide range of positions from various orientations. Therefore, the continuum robot gives physicians a wider addressable area and more approaches for access by a medical instrument (e.g., biopsy forceps, a fine needle, an aspiration needle, an ablation probe).

Also, the third bendable segment can be deformed by external forces to follow the shape of tortuous pathways in an anatomy, such as lung airways, blood vessels, and brain ventricles, while minimizing the force that is exerted on the anatomy. Therefore, by following the shape of the anatomy, the third bendable segment can be navigated by the first and the second bendable sections when the sheath is inserted, and the third bendable segment can develop the delivery line for the medial instruments (for medical treatments) and the driving force for controlling the sheath.

Moreover, because the length of the third bendable segment can be longer (e.g., twice as long) than the length of the first and the second bendable segments, the first and the second bendable segments can access deep-seated lesions in the anatomy and can have a localized motion area at the distal end of the sheath.

Also, the continuum robot can provide consistent and accurate distal maneuvering by having the first, the second, and the third bendable segments. The third bendable segment detaches the motions of the first and the second bendable segments from the rest of the proximal part of the sheath. The proximal part of the sheath, which includes the third bending bendable segment, goes through tortuous pathways in the anatomy and interacts with the anatomy with many contact points along the pathways, and those contact points interfere with the motion of the sheath and deteriorate the control accuracy of the sheath. Moreover, this deterioration in itself is not systematic but random because the contact points and degree of contact change by patient motions and the sheath maneuvers. Therefore, by detaching the first and the second bendable segments, the sheath can prevent the deterioration of the control and achieve consistent and accurate distal maneuvers.

Additionally, by having a single sheath instead of a nested assembly of multiple bendable elements, the sheath can increase a size of a tool channel while minimizing a size of an outer diameter. Also, the sheath can avoid the small gap between the multiple bendable elements, which reduces the risk that body liquids, such as blood, will cause a malfunction of the sheath.

Some embodiments have a third bendable segment that is ten or more times longer than the first and the second bendable segments. Thus, in such embodiments the sheath can access a further deep-seated lesion in the anatomy with improved distal dexterity using the first and the second bendable segments.

In embodiments that have a longer second bendable segment than the first bendable segment, the proximal end of the first bendable segment can be positioned in a wide range of areas by means of the bending angle of the second bendable segment. Furthermore, the shorter first bendable segment provides a wider orientation range in a smaller space of movement. Therefore, the first and the second bendable segments provide distal dexterity to a wide range of motions and orientations.

With a length ratio of 1:1 to 1:5 between the first and the second bendable segments, the first and the second bendable segments may provide distal dexterity with a better balance of range of motion and orientation for lung applications. In addition, a length ration between the second bendable segment and third bendable segment is optimal between 1:2 to 1:20. A target lesion in a lung application is often located in sub-segmental airways. The first bendable segment can be inserted into the sub-segmental airways, while the majority of the second bendable segment stays in the segmental airways, and also the distal end of the third bendable segment may deployed in the proximal end of the segmental airways. Therefore, it may be beneficial for the first, the second, and the third bendable segments to respectively have similar lengths to each generation of airways. In this particular embodiment, intended for use in the lungs, a length ratio between the first bendable segment, the second bendable segment, and the third bendable segment of 1:2:17, respectively, was found to be ideal. As can be imagined, the length ratios may be adjusted for use in various cavities based on the necessary/desired lengths.

By having an identical diameter in the first and the second bendable segments, the sheath does not include notches, steps, or slopes on the lateral surface of the sheath. Therefore, the sheath avoids a stress-concentration area, which generates a risk of malfunction, and avoids areas that cause high stress to the anatomy.

Also, the sheath can decrease physical interaction with the anatomy by avoiding surface features that potentially catch, bite, or scratch the anatomy, especially at the bifurcation point. Thus, the sheath can avoid trauma to the anatomy as well as unnecessary dislocation of the anatomy. Moreover, the sheath can reduce a risk of improper sterilization because it has a lateral surface with no notches, steps, or slopes.

The first and the second bendable segments independently change the bending angle and the bending plane in a three-dimensional space. By changing the bending angle and the bending plane, the first and the second bendable segments can direct their respective distal ends to all orientations within a maximum movable area in the three-dimensional space without causing a rotational motion of the sheath body around the sheath centerline.

By dislocating the distal end of the sheath three-dimensionally while maintaining a substantially constant orientation of the distal end, the sheath can target different positions within the target lesion. Because the orientation is maintained among those different positions, the sheath can deliver the medical instrument through the tool channel accurately even when the lesion is away from, is moving from, and is moving to the distal end of the sheath. Therefore, the sheath can provide detailed information about the lesion during a biopsy or make a large or unique-shaped ablation volume by combining the ablation from those different positions.

Also, by using this motion during a biopsy, the sheath can randomize the position within a planned range to increase the chances of hitting the lesion among multiple attempts.

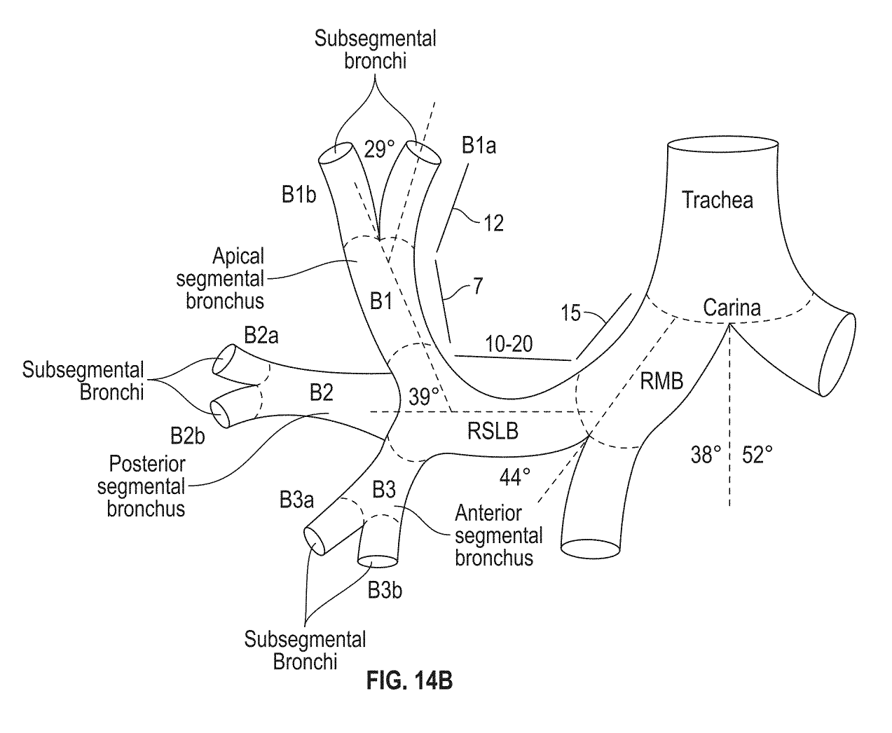

A planar phantom model 5o, as shown in FIG. 14A, was constructed to test the subject continuum robot 1, wherein the phantom model 50 is designed to represent the right upper lobe (RUL) of the lung. FIG. 14B provides an image of the lung portion being modeled in FIG. 14A. The model was chosen to depict only the RUL, because it is possible to observe in previous studies, that the RUL is the most common area for pulmonary nodules and represents a difficult inner cavity inaccessible to existing medical instruments. The phantom model 50 of the RUL of the lung is composed of two independent structures: the bronchi part 51 and the trachea part 52, as shown in FIG. 14A. The trachea part 52 represents the first part of a transbronchiol biopsy procedure (TBB). During a TBB procedure, to access the peripheral area of the lung, the sheath 3 is inserted via the trachea 53 until, for this study, the beginning of the right main bronchus (RMB) 54 and to the right segmental lobar bronchus (RSLB) 55. The relative large diameter of the sheath 3 compared to the diameters of the bronchi after the RSLB 55, makes the insertion of the sheath 3 deeper in the lung very challenging and difficult.

The bronchi part 51 of the phantom model 50 represents the second part of a TBB procedure. After the sheathe 3 reaches the RSLB 55, it is further extended to go deeper in the bronchi part 51 of the phantom 50. As mentioned earlier, the trachea part 52 and the bronchi part 51 are two independent structures of the lung phantom model 5o. The separation was made to avoid any interferences between the two parts. A force sensor is then incorporated and attached to the bottom of the bronchi part 51 to measure precisely the forces applied to the bronchi of the phantom 50. The different bronchi are numbered from B1a to B3b.

In total, 314 data points were measured to determine the efficacy of the subject continuum robot 1 and sheath 3. Deviation from the preplanned center line (path) per one robotic catheter placement to a target was collected and compared to know deviation data collected in previous experiments using manual catheters in the same phantom model. On average, the subject continuum robot 1 deviated 0.94 mm (SD=0.50 mm) while a manual catheter deviated 1.86 mm (SD=0.74 mm) with p-value of less than 0.01 indicating statistically extremely significant difference between the two group.

Moreover, an average force applied to the wall of the model 50 of 0.94 N (SD=0.30 N) was found for the manual catheter, and an average force of 0.13 N (SD=0.11 N) was found for the continuum robot 1. The P value for the two groups was less than 0.01. By conventional criteria, this difference is considered to be extremely statistically significant.

1. A medical apparatus comprising:
a driving unit;
a single sheath that includes a first bendable segment, a second bendable segment, and a third bendable segment, which are bendable by the driving unit; and
a controller configured to send a control signal to the driving unit for bending the at least three bendable segments,
wherein a length ratio between the first bendable segment and second bendable segment is between 1:1 to 1:5.

2. The apparatus of claim 1, wherein the first bendable segment and the second bendable segments are distal to the third bendable segment, and the first bendable segment and the second bendable segment are configured to be three-dimensionally bent by the driving unit.

3. The apparatus of claim 1, wherein the third bendable segment may be at least twice as long as each of the first and the second bendable segments, and the third bendable segment may be deformable by external force so that the third bendable segments can have a shape with at least one inflection point.

4. The apparatus of claim 1, wherein the driving unit is configured to apply a force to the third bendable segment in order to bend or maintain an orientation of a distal end of the third bendable segment while the external force is applied.

5. The apparatus of claim 1, wherein the controller is configured to control the first bendable segment and the second bendable segment independently of the third bendable segment, while a force is applied from the controller to the third bendable segment via the driving unit in order to maintain a shape of the third bendable segment.

6. The apparatus of claim 1, wherein the length of the first bendable segment and the second bendable segment is less than one tenth of the length of the third bendable segment.

7. The apparatus of claim 1, wherein a length ratio between the second bendable segment and third bendable segment is between 1:2 to 1:20.

8. The apparatus of claim 1, wherein a length ratio between the first bendable segment, the second bendable segment, and the third bendable segment is 1:2:17, respectively.

9. The apparatus of claim 1, wherein the diameters of the first bendable segment and the second bending segment are identical.

10. The apparatus of claim 1, wherein the first bendable segment and the second bendable segment are configured to independently change a respective bending angle and a respective bending plane in a three dimension space.

11. The apparatus of claim 1, wherein the controller is configured to dislocate the distal end of the single sheath three-dimensionally while keeping a substantially constant orientation of the distal end of the single sheath by bending the first bendable segment and the second bendable segment.

12. The apparatus of claim 1, wherein the sheath further comprises an outer wall covering at least the first bendable segment.

13. The apparatus of claim 12, wherein the outer wall is configured to attach to the first bendable segment and provide flexible support to the sheath.

14. The apparatus of claim 1, wherein at least the first bendable segment comprises a plurality of guide rings distributed along the longitudinal direction of the sheath.

15. The apparatus of claim 14, wherein the plurality of guide rings distributed along the longitudinal direction of the sheath create cavities between each of the guide rings.

16. The apparatus of claim 14, wherein the plurality of guide rings distributed along the longitudinal direction of the sheath are flexibly supported by an outer wall.
